(19) United States
(12) Patent Application Publication
(10) Pub. No.: US 2023/0154615 A1
(43) Pub. Date: May 18, 2023

(54) Experts Consulted System and Method for Pregnancy, Childbirth and Parenting

(71) Applicant: Freemom Technology Co., Ltd., Taipei City (TW)

(72) Inventor: Shyh-Jer Huang, Tainan City (TW)

(21) Appl. No.: 17/953,416

(22) Filed: Sep. 27, 2022

(30) Foreign Application Priority Data
Nov. 15, 2021 (TW) 110141389

(51) Int. Cl. G16H 50/20; G16H 10/60; G16H 40/63

(52) U.S. Cl. CPC G16H 50/20; G16H 10/60; G16H 40/63

(57) ABSTRACT

Present invention is related to an expert consulted system for pregnancy, childbirth and parenting which could be executed by a smart device. The smart device comprises the expert consulted system having a servo processing module, a data center and an expert consulted module. As users inputting their own personal data into the expert consulted system, the system will compare and analysis these data with the data center and the expert consulted module and output suitable pregnancy, childbirth and parenting advices or suggestions back to the smart device showing to the users.

### FIELD OF THE INVENTION

The present invention relates to an expert consulted system and a method of execution the same, more particularly, an intelligent system and method for providing online expert consultancy to pregnant women, childbirth, and parenting.

### BACKGROUND OF THE INVENTION

In the past when the birthrate was high, most women learned from their grandmother or from their own experience with multiple births in regard to pregnancy, childbirth, feeding their newborns, and even the subsequent raising of their babies. But nowadays, with fewer children, modern working women, and other factors, it is no longer able to rely on such traditional methods and it needs a more effective and more convenient way to assist women nowadays to smoothly get through the processes of birth and subsequent feeding and parenting.

With the popularity of smart devices in recent years, everyone owns their own mobile phone, tablet, computer, and high-speed Internet, and people's life is unable to leave these smart devices. Therefore, if information about women's pregnancy and childcare processes can be collected and processed properly, and useful information and advice can be given by a smart system, it will definitely bring great convenience to women.

### SUMMARY OF THE INVENTION

In order to overcome the various challenges that women may face in the processes of pregnancy and parenting, and suit the fact of popular use of smart devices nowadays, the primary objective of the present invention is to provide experts consulted system and method for pregnancy, childbirth, and parenting, which comprises a pregnancy and parenting system, a servo processing module, a data center, or an expert consulted module, executed by a smart device with one or more of any combinations of those that interact with each other with the messages or signals.

Wherein, the system includes a digital lactation assistant module, a pregnancy expert module, an infant behavior assessment module, and a basic information module for collecting information input by the user and transmitting it to the servo processing module, then sending it to the data center for comparison and analysis.

Wherein, the data center contains a lactation information database, a pregnancy information database, and an infant behavior information database. The data center compares and analyzes the incoming user's information in the databases and transmits the results to the expert consulted module.

Wherein, the expert consulted module contains a lactation assistant/feeding expert module, and it further contains several lactation assistant or feeding expert accounts to receive the results of the comparison and analysis from the lactation information database, the pregnancy information database, and the infant behavior information database in the data center, and create a feedback of a lactation advice information and/or feeding advice information to the pregnancy and parenting system and display it to the user on the smart device.

Wherein, the data center receives information obtained by the pregnancy and parenting system which is on a continuous basis, and the lactation advice and/or feeding advice information given by the expert consulted module will be adjusted according to the trends obtained from the continuous information.

Wherein, the digital lactation assistant module receives lactation information input by the user, including frequency, duration, volume, color, or texture of breast milk; the pregnancy expert module receives pregnancy information input by the user, including pregnancy period, expected delivery date, or nutritional status; the infant behavior assessment module receives infant behavior information input by the user, including infant feeding time, crying status, learning status or multiple growth and development statuses; and the basic information module receives physical quality basic information input by the user, including height, weight, age and gender of pregnant women and their infants.

Wherein, the lactation information database stores several categories of lactation data and advice information, and the digital lactation assistant module compares and analyzes the information obtained to the lactation information database via the servo processing module; the pregnancy information database stores several categories of pregnancy data and advice information, and the pregnancy expert module compares and analyzes the information obtained to the pregnancy information database via the servo processing module; and the infant behavior information database stores several categories of infant behavior data and advice information, and the infant behavior assessment module compares and analyzes the information obtained to the infant behavior information database via the servo processing module.

Wherein, the expert consulted module further comprises an artificial intelligence comparison module, the lactation and/or the feeding advice information is collected by the artificial intelligence comparison module, then transmitted to and recorded in the corresponding lactation information database, pregnancy information database, and infant behavior information database, and continuous learning and updating from the collected information, the comparison results, and the outcome of the assessment and analysis.

Wherein, the pregnancy and parenting system is electrically signaled to an external sensor terminal, and the inside of the pregnancy and parenting system is equipped with an additional sensor terminal data linkage module.

Wherein, the data center further contains a physique information database, correspondingly, the expert consulted module further contains a lactation/feeding/physique expert module, and the advice information fed back from the expert consulted module further contains the information of physique advice which includes pelvic toning or abdominal muscle training.

Further, another object of the present invention is to provide a method for executing the pregnancy and childbirth expert consulted service corresponding to the aforementioned system, the steps of which comprise: information inputting and generating: inputting and generating one or more of any combination of a lactation information, a pregnancy information, an infant behavior information and/or a physical quality basic information into a pregnancy and parenting system; information receiving and transmitting: the pregnancy and parenting system receives the aforementioned lactation information, pregnancy information, infant behavior information and/or physical quality basic information and transmits them to a data center via the servo processing module; information processing, comparing and analyzing: the data center stores the data and advice information of the lactation, pregnancy and/or infant behavior, and proceeds with the comparing and analyzing of the input and received lactation, pregnancy, infant behavior and/or the physical quality basic information, then transmits the results to the expert consulted module; and output an advice information: the expert consulted module is used to receive the aforementioned information compared and analyzed by the data center and generate a lactation advice and/or a feeding advice information message.

Preferably, the lactation advice information and/or the feeding advice information is further collected by the artificial intelligence comparison module and transmitted to and recorded in the corresponding data center as described above, and continuous learning and updating from the collected information, the comparison results, and the outcome of the assessment and analysis.

Preferably, the data center receives the information obtained by the pregnancy and parenting system which is on a continuous basis, and the lactation advice and/or feeding advice information given by the expert consulted module will be adjusted according to the trends obtained from the continuous information.

Preferably, the expert consulted module contains several lactation assistant or feeding expert accounts to receive the information of the comparison and analysis from the data center.

By the above description, it can be seen that the present invention has the following beneficial effects and advantages.

1. The present invention assists pregnant women and mothers in solving various problems during the growth of their babies more easily and precisely, and assists them in their infant feeding and nurturing behaviors and the needs before, during, and after pregnancy by integrating smart devices and databases together with digital lactation assistants and online consultation with experts, and so forth.

2. The main features of the pregnancy and childbirth expert consulted system provided by the present invention are the digital lactation assistant and expert online consultation, as well as the continuous information collection on the time-axis, and the assessment and advice given can be adjusted and fed back according to the continuous trends collected, to accomplish a more precise assessment and advice.

### BRIEF DESCRIPTION OF THE DRAWINGS

The present invention will be further illustrated by way of exemplary embodiments, which will be described in detail through the accompanying drawings. These embodiments are not limiting, and in these embodiments, the same numbers refer to the same structures, wherein:

FIG. 1 is a schematic diagram of the first preferred embodiment of an expert consulted system for pregnancy, childbirth and parenting of the present invention;

FIG. 2 is a schematic diagram of the information trend on which the lactation advice information and/or the feeding advice information is given by the expert consulted system for pregnancy, childbirth and parenting of the present invention;

FIG. 3 is a schematic diagram of the second preferred embodiment of the expert consulted system for pregnancy, childbirth and parenting of the present invention;

FIG. 4 is a schematic diagram of the third preferred embodiment of the expert consulted system for pregnancy, childbirth and parenting of the present invention; and

FIG. 5 is a flowchart of the first preferred embodiment of a method of the expert consulted system for pregnancy, childbirth and parenting of the present invention.

### DESCRIPTION OF THE PREFERRED EMBODIMENTS

In order to more clearly illustrate the technical solutions of the embodiments of the present invention, the following is a brief description of the accompanying drawings that are required for use in describing the embodiments. Obviously, the drawings in the following description are only some examples or embodiments of the present invention, it is possible for those persons who are with ordinary skills in the art to apply the present invention to other similar situations based on these drawings without creative effort. Unless obvious from the linguistic context or otherwise noted, the same numbers in the drawings represent the same structures or operations.

One should understand that the terms “system”, “device”, “unit” and/or “module” as used herein is a method used to distinguish different components, elements, parts, sections or assemblies at different levels. However, if other terms can serve the same purpose, said terms may be substituted by other expressions.

As shown in the present invention, unless the context clearly suggests otherwise, the words “a” and/or “the” do not refer specifically to the singular, but may also include the plural. In general, the terms “include” and “contain” suggest only the inclusion of clearly identified steps and elements that do not constitute an exclusive list, and the method or apparatus may also contain other steps or elements.

### An Expert Consulted System for Pregnancy, Childbirth and Parenting.

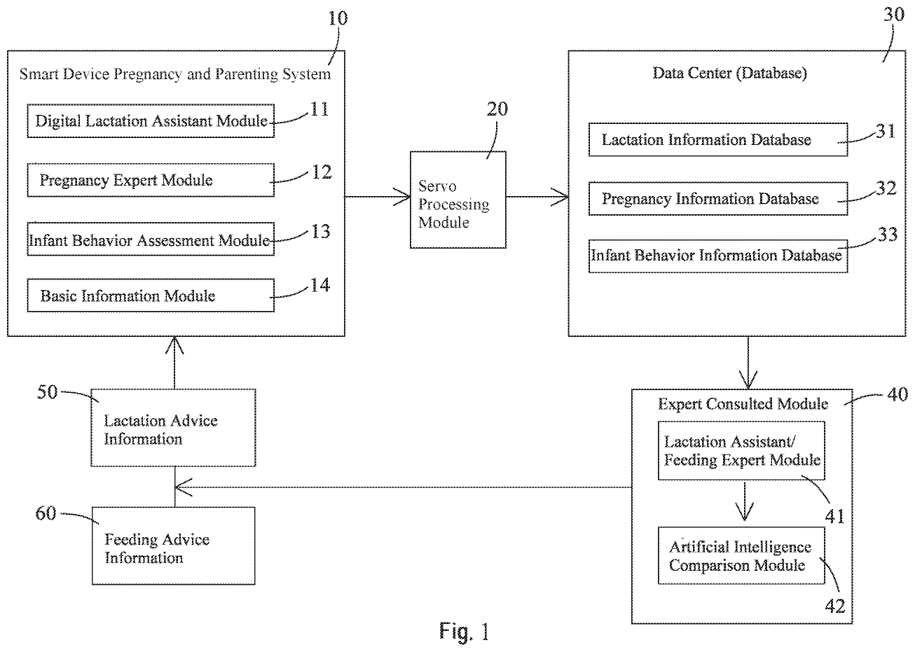

Referring to FIG. 1, the first preferred embodiment of the expert consulted system for pregnancy, childbirth and parenting of the present invention, comprising: a pregnancy and parenting system 10, a servo processing module 20, a data center (or database) 30, and an expert consulted module 40, executed by a smart device, and interacting with each other with messages or signals, preferably using wired or wireless transmission of such signals or messages, including any form such as wired network, wireless network, or Bluetooth.

### The Pregnancy and Parenting System.

The pregnancy and parenting system 10 contains one or more of any combination of a digital lactation assistant module 11, a pregnancy expert module 12, an infant behavior assessment module 13, or a basic information module 14.

The digital lactation assistant module 11 is used to receive lactation information input by users, including the frequency, duration, volume, color, or texture, etc., of breast milk.

This pregnancy expert module 12 is used to receive pregnancy information input by the user, including physical and mental information of pregnant women before and after the pregnancy, such as the duration of pregnancy, expected delivery date or nutritional status, etc.

The infant behavior assessment module 13 is used to receive infant behavior information input by users, including infant feeding time, infant crying status, infant learning status, or multiple growth and development statuses, etc.

The basic information module 14 is used to receive the physical quality basic information input by users, including height, weight, age, and gender of pregnant women and their infants.

The user information collected by the pregnancy and parenting system 10 will be transmitted to the servo processing module 20, then the servo processing module 20 will send it to the data center 30 for further comparison and analysis.

### The Data Center (Database).

The data center 30 contains at least one or more combinations of a lactation information database 31, a pregnancy information database 32, or an infant behavior information database 33.

The lactation information database 31 stores several categories of lactation data and advice information, and the digital lactation assistant module 11 compares and analyzes the information obtained to the lactation information database 31 via the servo processing module 20.

The pregnancy information database 32 stores several categories of pregnancy data and advice information, and the pregnancy expert module 12 compares and analyzes the information obtained to the pregnancy information database 32 via the servo processing module 20.

The infant behavior information database 33 stores several categories of infant behavior data and advice information, and the infant behavior assessment module 13 compares and analyzes the information obtained to the infant behavior information database 33 via the servo processing module 20.

The data center 30 transfers the all results of comparison and analysis from the database to the expert consulted module 40. The data center 30 may receive the information obtained by the aforementioned pregnancy and parenting system 10 intermittently or continuously, but preferably, it collects the information continuously and transfers it to the expert consulted module 40.

### The Expert Consulted Module.

The expert consulted module 40 contains a lactation assistant/feeding expert module 41 and an artificial intelligence comparison module 42.

The lactation assistant/feeding expert module 40 may preferably contain several lactation assistant or feeding expert accounts to receive the results of the comparison and analysis from the lactation information database 31, the pregnancy information database 32, and the infant behavior information database 33 in the data center 30, and to generate feedback with lactation advice information 50 and/or feeding advice information 60 to the pregnancy and parenting system 10 and display them to the user on the smart device.

The lactation advice information 50 includes the plans, adjustments, or advice on lactation, and the feeding advice information 60 includes the advice on parenting, usage of products (optional) and courses, etc.

Preferably, the lactation advice information 50 and/or the feeding advice information 60 generated by the lactation assistant/feeding expert module 40 will be collected by the artificial intelligence comparison module 42, and transmitted to and recorded in the corresponding lactation information database 31, pregnancy information database 32, and infant behavior information database 33, and continuous learning and updating from the collected information, the comparison results, and the outcome of the assessment and analysis.

On the other hand, since the method of collecting information in the present invention is continuous on the time-axis, the lactation advice information 50 and/or the feeding advice information 60 given by the expert consulted module 40 will be adjusted and provided according to the trend.

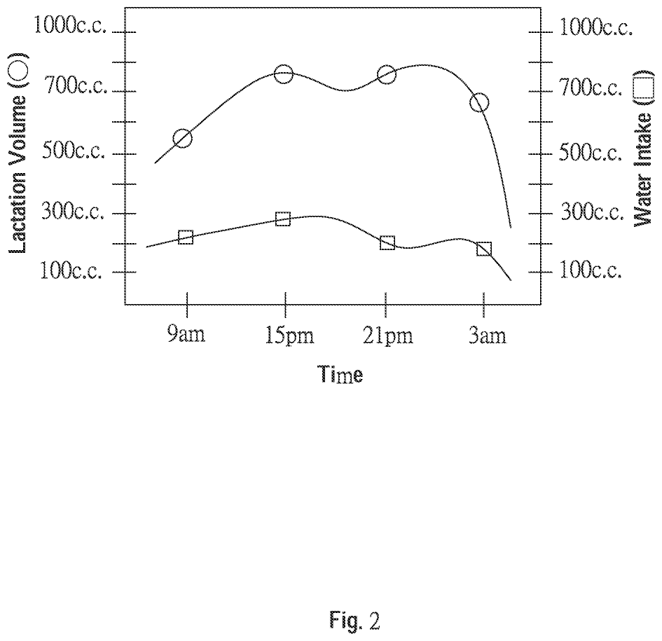

More specifically, the so-called lactation advice information 50 and/or the feeding advice information 60 will be carried out according to a trend, for example, the user continuously inputs the required lactation information, pregnancy information, infant behavior information or physical quality basic information on the time-axis, and the data center 30 and the expert consulted module 40 of the present invention will actively compare and adjust the advice and inferences given according to the variation of information on the time-axis. For example, the continuous time-axis shown in FIG. 2, where a user's lactation volume and water intake are fluctuating in correlation, the data center 30 and the expert consulted module 40 will then collect the information based on this time variation to infer and give advice and assessment results to the user to suggest to drink more water.

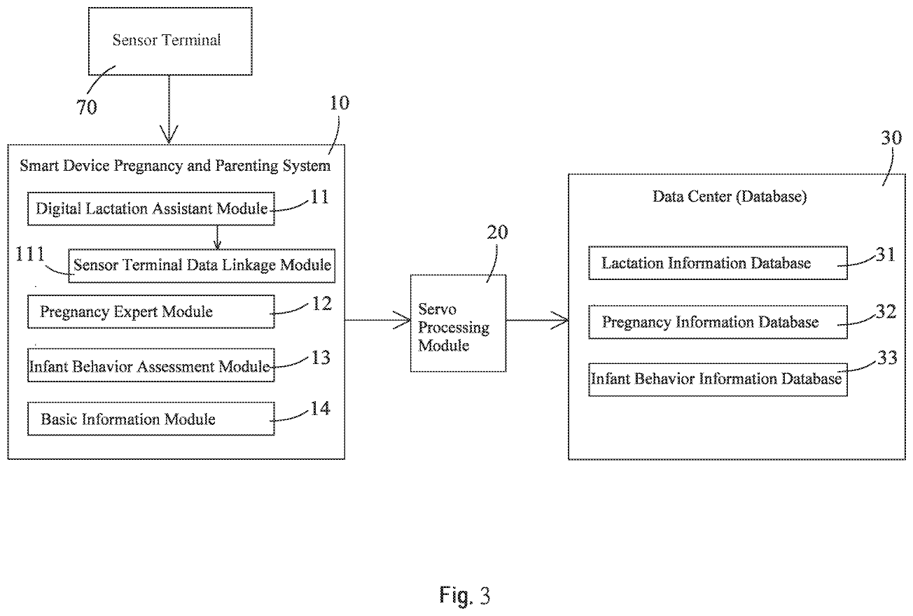

Referring to FIG. 3, the second preferred embodiment of the expert consulted system for pregnancy, childbirth and parenting is essentially the same as the first preferred embodiment described above, except that the pregnancy and parenting system 10 is electrically signaled to an external sensor terminal 70, and an additional sensor terminal data linkage module 111 is provided inside of the pregnancy and parenting system 10.

A preferred embodiment of the sensor terminal 70 can be a breast pump, nursing bottle, or other devices with a sensing function. The user uses the sensor terminal 70 for breast pumping or breastfeeding, and the sensor terminal 70 has a sensor that continuously senses information data such as the amount of breast pumping or breastfeeding, and transmits the sensed data back to the pregnancy and parenting system 10.

As to the sensor terminal data linkage module 111 will continuously collect the external information sensed by the sensor terminal 70 and transmit it via the servo processing module 20 to the data center 30 and the expert consulted module 40, with the same method as the first preferred embodiment above, to feed back the lactation advice information 50 and/or the feeding advice information 60 to the user.

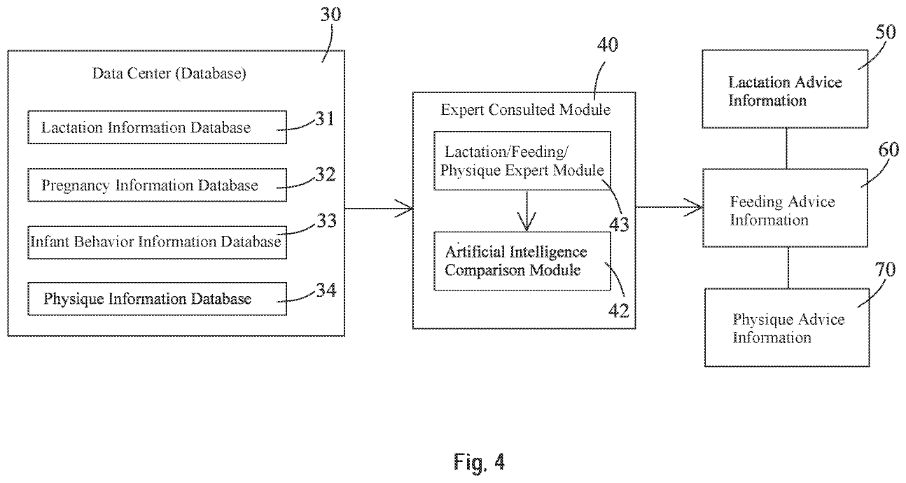

Referring to FIG. 4, the third preferred embodiment of the expert consulted system for pregnancy, childbirth and parenting is essentially the same as the first preferred embodiment described above, in that the data center 30 further contains a physique information database 34. Also, the lactation assistant/feeding expert module 41 of the expert consulted module 40 is further updated as a lactation/feeding/physique expert module 43, whereas the advice information fed back by the expert consulted module 40 further contains a physique advice information 70. The physique information database 34 is used to receive physique information input by the user, including pelvic status, muscle mass and muscle growth status, such as abdominal muscle growth status, etc.

The physique information database 34 transmits the results of the comparison and analysis to the expert consulted module 40 where the lactation/feeding/physique expert module 43 compares the analyzed results and provides feedback with the physique advice information 70, such as pelvic toning advice or abdominal muscle training advice, etc.

### A Method of Executing the Expert Consulted System for Pregnancy, Childbirth and Parenting.

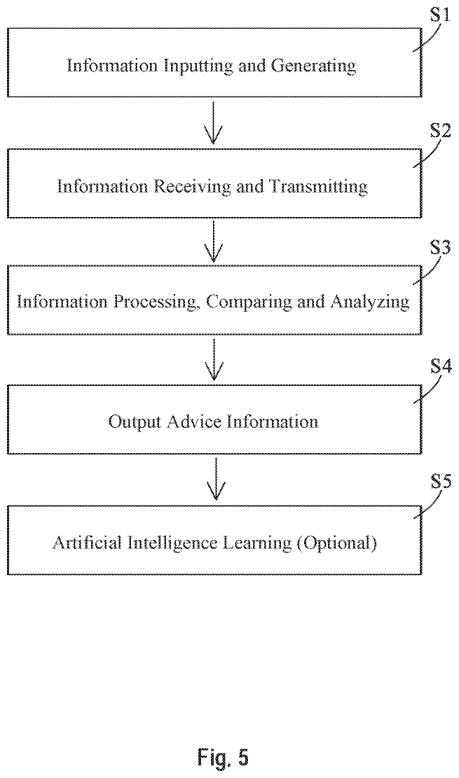

Referring to FIG. 5, the present invention provides a method for executing the aforementioned expert consulted system, with steps comprising:

S1: information inputting and generating: one or more of any combination of the lactation information, the pregnancy information, the infant behavior information and/or the physical quality basic information is input and generated into the pregnancy and parenting system 10;

S2: information receiving and transmitting: the pregnancy and parenting system 10 receives the lactation information, the pregnancy information, the infant behavior information and/or the physical quality basic information, and transmits them to the data center 30 via the servo processing module 20;

S3: information processing, comparing and analyzing: the data center 30 stores the data and advice information of the lactation, pregnancy and/or infant behavior, and proceeds with the comparing and analyzing of the input and received lactation, pregnancy, infant behavior and/or physical quality basic information, then transmits the results to the expert consulted module 40; whereas, the data center 30 may receive the information obtained by the pregnancy and parenting system 10 either intermittently or continuously;

S4: output advice information: the expert consulted module 40 contains several lactation assistant or feeding expert accounts to receive the information of the comparison and analysis from the data center 30 and generate the lactation advice information 50 and/or the feeding advice information 60; and

S5: artificial intelligence learning (optional): the lactation advice information 50 and/or the feeding advice information 60 is collected by the artificial intelligence comparison module 42 and then transmitted to and recorded in the corresponding data center 30, and continuous learning and updating from the collected information, the comparison results, and the outcome of the assessment and analysis.

Finally, it should be understood that the embodiments described in the present invention are only used to illustrate the principles of the embodiments of the present invention, other variations may also fall within the scope of the present invention. Therefore, as an example and not as a limitation, alternative configurations of the embodiments of the present invention may be considered to be consistent with the teachings of the present invention. Accordingly, the embodiments of the present invention are not limited to those embodiments expressly introduced and described.

What is claimed is:

1. An expert consulted system for pregnancy, childbirth and parenting, comprising: a pregnancy and parenting system, a servo processing module, a data center, and an expert consulted module interconnected with each other with messages or signals, wherein:
the pregnancy and parenting system contains one or more combinations of a digital lactation assistant module, a pregnancy expert module, an infant behavior assessment module, or a basic information module, and is used for collecting information input by the user and transmitting it to the servo processing module, then sending it to the data center for comparison and analysis;
the data center contains one or more combinations of a lactation information database, a pregnancy information database, or an infant behavior information database; the data center compares and analyzes the incoming user information in all of its databases and transmits the results to the expert consulted module; and
the expert consulted module contains a lactation assistant/feeding expert module that receives the results of the comparison and analysis from the lactation information database, the pregnancy information database, and the infant behavior information database in the data center, and generates feedback with lactation advice information and/or feeding advice information to the pregnancy and parenting system and display the information to the user on the smart device.

2. The expert consulted system for pregnancy, childbirth and parenting according to claim 1, wherein: the data center receives information obtained by the pregnancy and parenting system which is on a continuous basis, and the lactation advice and/or feeding advice information given by the expert consulted module will be adjusted according to the trends obtained from the continuous information.

3. The expert consulted system for pregnancy, childbirth and parenting according to claim 1, wherein:
the digital lactation assistant module contains several lactation assistant or feeding expert accounts for receiving lactation information input by the user, including frequency, duration, volume, color, or texture of breast milk;
the pregnancy expert module is used to receive pregnancy information input by the user, including the duration of pregnancy, expected delivery date, or nutritional status;
the infant behavior assessment module is used to receive infant behavior information input by the user, including infant feeding time, infant crying status, infant learning status, or multiple growth and development statuses; and
the basic information module is used to receive physical quality basic information input by the user, including height, weight, age, and gender of pregnant women and their infants.

4. The expert consulted system for pregnancy, childbirth and parenting according to claim 2, wherein:
the digital lactation assistant module contains several lactation assistant or feeding expert accounts for receiving lactation information input by the user, including frequency, duration, volume, color, or texture of breast milk;
the pregnancy expert module is used to receive pregnancy information input by the user, including the duration of pregnancy, expected delivery date, or nutritional status;
the infant behavior assessment module is used to receive infant behavior information input by the user, including infant feeding time, infant crying status, infant learning status, or multiple growth and development statuses; and
the basic information module is used to receive physical quality basic information input by the user, including height, weight, age, and gender of pregnant women and their infants.

5. The expert consulted system for pregnancy, childbirth and parenting according to claim 1, wherein:
the lactation information database stores several categories of lactation data and advice information, and the digital lactation assistant module compares and analyzes the information obtained to the lactation information database via the servo processing module;
the pregnancy information database stores several categories of pregnancy data and advice information, and the pregnancy expert module compares and analyzes the information obtained to the pregnancy information database via the servo processing module; and
the infant behavior information database stores several categories of infant behavior data and advice information, and the infant behavior assessment module compares and analyzes the information obtained to the infant behavior information database via the servo processing module.

6. The expert consulted system for pregnancy, childbirth and parenting according to claim 2, wherein:
the lactation information database stores several categories of lactation data and advice information, and the digital lactation assistant module compares and analyzes the information obtained to the lactation information database via the servo processing module;
the pregnancy information database stores several categories of pregnancy data and advice information, and the pregnancy expert module compares and analyzes the information obtained to the pregnancy information database via the servo processing module; and
the infant behavior information database stores several categories of infant behavior data and advice information, and the infant behavior assessment module compares and analyzes the information obtained to the infant behavior information database via the servo processing module.

7. The expert consulted system for pregnancy, childbirth and parenting according to claim 1, wherein: the expert consulted module further comprises an artificial intelligence comparison module, wherein the lactation advice and/or the feeding advice information is collected by the artificial intelligence comparison module and then transmitted to and recorded in the corresponding lactation information database, pregnancy information database, and infant behavior information database, and continuous learning and updating from the collected information, the comparison results, and the outcome of the assessment and analysis.

8. The expert consulted system for pregnancy, childbirth and parenting according to claim 2, wherein: the expert consulted module further comprises an artificial intelligence comparison module, wherein the lactation advice and/or the feeding advice information is collected by the artificial intelligence comparison module and then transmitted to and recorded in the corresponding lactation information database, pregnancy information database, and infant behavior information database, and continuous learning and updating from the collected information, the comparison results, and the outcome of the assessment and analysis.

9. The expert consulted system for pregnancy, childbirth and parenting according to claim 1, wherein: the pregnancy and parenting system is electrically signaled to an external sensor terminal, and an additional sensor terminal data linkage module is provided inside of the pregnancy and parenting system, wherein:
the sensor terminal has a sensor that senses and acquires lactation information, pregnancy information, infant behavior information and/or physical quality basic information, and transmits the sensed data back to the pregnancy and parenting system.

10. The expert consulted system for pregnancy, childbirth and parenting according to claim 1, wherein: the data center further contains a physique information database, correspondingly, the expert consulted module further contains a lactation/feeding/physique expert module, and the advice information feedback from the expert consulted module further contains information of physique advice which includes information of pelvic toning or abdominal muscle training.

11. The expert consulted system for pregnancy, childbirth and parenting according to claim 2, wherein: the data center further contains a physique information database, correspondingly, the expert consulted module further contains a lactation/feeding/physique expert module, and the advice information feedback from the expert consulted module further contains information of physique advice which includes information of pelvic toning or abdominal muscle training.

12. A method of executing the expert consulted system for pregnancy, childbirth and parenting, as described in any of claim 1, comprising steps of:
information inputting and generating: one or more of any combination of the lactation information, the pregnancy information, the infant behavior information, or the physical quality basic information is input and generated into the pregnancy and parenting system;
information receiving and transmitting: the pregnancy and parenting system receives the lactation information, the pregnancy information, the infant behavior information and/or the physical quality basic information and transmits them to the data center via the servo processing module;
information processing, comparing and analyzing: the data center stores one or more of any combination of the data and advice information of lactation, pregnancy and/or infant behavior, and proceeds with the comparing and analyzing of the input and received lactation, pregnancy, infant behavior and/or physical quality basic information, then transmits the results to the expert consulted module; and
output advice information: the expert consulted module receives the information of the comparison and analysis by the data center and generates the lactation advice information and/or the feeding advice information.

13. The method of executing the expert consulted system for pregnancy, childbirth and parenting, according to claim 12, wherein: the lactation advice information and/or the feeding advice information is further collected by the artificial intelligence comparison module and transmitted to and recorded in the corresponding data center, and continuous learning and updating from the collected information, the comparison results, and the outcome of the assessment and analysis.

14. The method of executing the expert consulted system for pregnancy, childbirth and parenting, according to claim 12, wherein: the data center receives information obtained by the pregnancy and parenting system which is on a continuous basis, and the lactation advice and/or feeding advice information given by the expert consulted module will be adjusted according to the trends obtained from the continuous information.

15. The method of executing the expert consulted system for pregnancy, childbirth and parenting, according to claim 13, wherein: the data center receives information obtained by the pregnancy and parenting system which is on a continuous basis, and the lactation advice and/or feeding advice information given by the expert consulted module will be adjusted according to the trends obtained from the continuous information.

16. The method of executing the expert consulted system for pregnancy, childbirth and parenting, according to claim 12, wherein: the expert consulted module contains several lactation assistant or feeding expert accounts to receive the information of the comparison and analysis from the data center.

17. The method of executing the expert consulted system for pregnancy, childbirth and parenting, according to claim 13, wherein: the expert consulted module contains several lactation assistant or feeding expert accounts to receive the information of the comparison and analysis from the data center.
